(19) United States
(12) Patent Application Publication
(10) Pub. No.: US 2009/0234269 A1
(43) Pub. Date: Sep. 17, 2009

(54) System for tissue electroporation and method for use thereof

(75) Inventor: Michael Tavger, Katzrin (IL)

Correspondence Address: Fay Sharpe LLP, 1228 Euclid Avenue, 5th Floor, The Halle Building, Cleveland, OH, 44115, US

(73) Assignee: TavTech Ltd.

(21) Appl. No.: 12/075,380

(22) Filed: Mar. 11, 2008

(51) Int. Cl. A61N 1/30; A61M 11/00

(52) U.S. Cl. 604/20

(57) ABSTRACT

A device for electroporation of tissue is provided. The device includes a mist jet delivery nozzle arrangement that is connected to a pressurized gas source and also connected to a liquid source. The device also includes a charge generator in electrical communication with the liquid provided by the liquid source wherein the generator supplies charge to the liquid. The delivery nozzle arrangement is arranged and operative to accelerate and emit the pressurized gas and to accelerate and emit the liquid as a mist of accelerated charged droplets. The charged droplets alight, and then electrically discharge, on the tissue being treated. A system including the device and a method for using the device and system are also discussed

### FIELD OF THE INVENTION

The present invention relates to a system for electroporation of tissue and a method for use thereof.

### BACKGROUND OF THE INVENTION

Electroporation, or electropermeabilization, is a method for significantly increasing the electrical conductivity and permeability of a cell plasma membrane resulting from an externally applied electrical field. It is usually used in molecular biology or medicine as a way of introducing some substance into a cell, such as a drug. Electroporation may be used in tumor treatment, in gene therapy, and in cell-based therapy.

Pores are formed when the voltage across a plasma membrane exceeds its dielectric strength. If the strength of the applied electrical field and/or duration of exposure to it are properly chosen, the pores formed by the electrical field pulses reseal after a short period of time, during which extracellular compounds have a chance to enter into the cell. However, excessive exposure of live cells to electrical fields can cause apoptosis and/or necrosis, processes that result in cell death.

Electroporation or electropermeabilization is a transitory structural perturbation of the lipid bilayer cell membranes of cells resulting from the application of high voltage pulses. Its application to tissue has been shown to increase transdermal drug delivery by several orders of magnitude. Moreover, electroporation, used alone or in combination with other methods, can expand the range of drugs which can be delivered transdermally. Molecular transport through transiently permeabilized tissue by electroporation results mainly from enhanced diffusion and electrophoresis.

Transdermal drug delivery offers several advantages over conventional routes. It avoids first-pass metabolism of the drug in the gastrointestinal tract. Transdermal delivery has the potential for sustained and controlled drug release. Moreover, it is a non-invasive mode of drug delivery with no trauma or risk of infection. Patient compliance may be improved by this user-friendly method.

In spite of the advantages of transdermal delivery, only a small percentage of drugs can be delivered transdermally due to the barrier properties of the tissue, principally the skin's stratum corneum. The difficulty with transdermal drug delivery is that the outermost layer of the skin, the stratum corneum, is a very effective barrier to the transport of hydrophilic or ionized species. Only small lipophilic drugs can be delivered at therapeutic rates by passive diffusion. For most drugs, times to reach steady-state fluxes across tissue barriers are measured in hours.

Because the stratum corneum is the main barrier to transdermal transport, the disruption of the stratum corneum and electroporation of its lipid bilayers can dramatically influence overall tissue permeability and it has been suggested that electroporation of its intercellular lipid bilayers might enhance percutaneous drug delivery. The stratum corneum contains approximately 100 bilayer membranes in series; electrical breakdown of these membranes are associated with a dramatic increase in transport which has been observed for applied voltages in the range of about 100-1500 V. This corresponds to a range of trans-membrane potentials used for electroporation in cells of about 0.3-1.0 V per bilayer. The electric field pulses are typically applied for durations of about 10 nanoseconds to about 10 milliseconds. Under these conditions reversible electrical breakdown and high molecular transport have been observed, resulting from structural rearrangements of the cell membrane. It has been hypothesized that these rearrangements consist of the formation of temporary aqueous pathways with the electric field inducing pore formation and providing a local driving force for molecular transport.

The main practical problem with electroporation is that electricity cannot pass through the stratum corneum and there is contact resistance in the area where the electrode contacts the tissue. Moreover, the high voltage pulsations used in electroporation do not correspond to the membrane's natural resonance frequency, especially in the typical non-uniform cell conglomeration of real tissue. Due to the design limitations of electrical circuits, most electroporation devices generate a range of uniform pulses that do not conform to the sensitive frequencies of the membranes. Cell density and different cell orientations also impact the efficacy of electroporation.

There remains a need for a relatively simple system and/or a device, and a method for its use, which would facilitate electroporation especially for use in drug therapy without destroying the tissue to which the high voltage has been applied. A device and/or system which would generate electrical pulses without current distortion is desirable. Similarly, a device and/or system and a method that would substantially reduce or eliminate tissue contact resistance would be desirable.

### SUMMARY OF THE INVENTION

It is an object of the present invention to provide a device, a system and a method that can be used for electroporation of tissue.

It is a further object of the present invention to provide a device, a system and a method for generating electrical pulses without current distortion. The device and system provide charged droplets which upon discharge generate pulses without current distortion that may arise from the conducting medium and electrodes.

It is an object of the present invention to provide an electrodeless system for electroporation, one where no electrodes need be attached to the tissue at the point of treatment.

It is a further object of the present invention to provide a device, a system and a method for reducing tissue contact resistance during electroporation.

In a first aspect of the present invention there is provided a device for electroporation of pre-selected tissue. The device includes:
a) a mist jet delivery nozzle arrangement having a distal end and a proximal end and the nozzle arrangement is connected to a pressurized gas source providing a pressurized gas and also connected to a liquid source; and
b) a charge generator in electrical communication with the liquid provided by the liquid source wherein the generator supplies charge to the liquid, and

wherein the delivery nozzle arrangement is arranged and operative to accelerate and emit the pressurized gas and also arranged and operative to similarly accelerate and emit the liquid as a mist of accelerated charged droplets, the charged droplets alighting and then electrically discharging on the pre-selected tissue thereby effecting electroporation.

In an embodiment of the device of the present invention, the charge generator provides charge to the liquid at a current of about 100 microamperes or less.

In yet another embodiment of the device, the mist jet delivery nozzle arrangement further includes:
i) one or more gas discharge nozzles arranged to receive a flow of pressurized gas from the gas source via a gas inlet port where the one or more gas discharge nozzles are configured to accelerate the flow of gas so as to emit it at an elevated velocity; and
ii) one or more liquid discharge nozzles arranged to receive a flow of liquid from the liquid source via a liquid inlet port and operative to emit the flow of liquid into the elevated velocity flow of gas, thereby to similarly accelerate the velocity of the discharged liquid as a mist of accelerated droplets.

In yet another embodiment of the device, the flow of gas entering the one or more gas discharge nozzles is at a pressure of a first magnitude, and the one or more gas discharge nozzles are operative to cause a pressure drop in the gas flow therethrough such that the pressure of the gas emitted from the one or more gas discharge nozzles is of a second magnitude. The first magnitude is at least twice the second magnitude, so as to cause a shock wave in the gas and liquid flow downstream of the one or more gas discharge nozzles and the one or more liquid discharge nozzles. The shock wave atomizes the liquid emitted from the one or more liquid discharge nozzles into a high velocity mist of droplets, forming a mist of droplets suspended in the flow of emitted high velocity gas.

In still another embodiment of the device, the device further includes an electrode positioned in the liquid source for charging the liquid therein with charge supplied by the charge generator.

In a further embodiment of the device, the device further includes an electrode positioned in the mist jet delivery nozzle for charging the liquid being delivered to, and emitted from, the nozzle. The electrode is in electrical communication with the charge generator.

In another aspect of the present invention, there is provided a system for electroporation of pre-selected tissue of a patient. The system includes:
a) a pressurized gas source;
b) a liquid source; and
c) a device which includes:
   i) a gas inlet port connected to the pressurized gas source;
   ii) a liquid inlet port connected to the liquid source;
   iii) a mist jet delivery nozzle arrangement having a distal end and a proximal end and including:
      1) one or more gas discharge nozzles arranged to receive a flow of pressurized gas from the gas inlet port and configured to accelerate the flow of gas so as to emit it at an elevated velocity; and
      2) one or more liquid discharge nozzles arranged to receive a flow of liquid from the liquid inlet port and operative to emit the flow of the liquid into the elevated velocity flow of gas at the distal end of the nozzle arrangement, thereby to similarly accelerate the emitted liquid as a mist of accelerated droplets;
   iv) a charge generator in electrical communication with the liquid so that the accelerated droplets carry charge supplied by the generator; and
   v) an electrical connector in electrical communication with the charge generator and attachable to the body of the patient, the system thereby allowing for the discharge of the electrically charged droplets when the droplets alight on the pre-selected tissue effecting electroporation therein.

In an embodiment of the system, the device further includes an electrode positioned in the liquid source for electrically charging the liquid therein with charge supplied by the charge generator.

In another embodiment of the system, the device further includes an electrode positioned in the mist jet delivery nozzle arrangement for charging the liquid being delivered to, and emitted from the nozzle. The electrode is in electrical communication with the charge generator.

In still another embodiment of the system, the flow of gas entering the one or more gas discharge nozzles is at a pressure of a first magnitude, and the one or more gas discharge nozzles is operative to cause a pressure drop in the gas flow therethrough such that the pressure of the gas emitted from the one or more gas discharge nozzles is of a second magnitude. The first magnitude is at least twice the second magnitude, so as to cause a shock wave in the gas and liquid flow downstream of the one or more gas discharge nozzles and the one or more liquid discharge nozzles. The shock wave atomizes the liquid emitted from the one or more liquid discharge nozzles into a high velocity mist of droplets, thereby to form a mist of droplets suspended in the flow of emitted high velocity gas.

In another embodiment of the present invention, there is provided a method for electroporation of a pre-selected area of tissue. The method includes the steps of:
a) accelerating a flow of a gas to an elevated velocity;
b) introducing into the elevated velocity gas flow a flow of electrically charged liquid, thereby to fragment the liquid into a mist of electrically charged droplets, and to accelerate the mist to an accelerated velocity similar to the velocity of the gas flow; and
c) exposing the pre-selected area of tissue to the accelerated electrically charged droplet mist.

In an embodiment of the method, the method further includes the step of providing charge to the liquid.

In another embodiment of the method, the charge provided to the liquid is provided at a current of about 100 microamperes or less.

In a further embodiment of the method, the step of accelerating a flow of a gas includes accelerating the gas through one or more gas discharge nozzles so as to provide a gas flow at an elevated velocity.

In still another embodiment of the method, the step of introducing into the elevated velocity gas flow a flow of an electrically charged liquid, includes the elevated gas flow being at a pressure of a first magnitude, and the one or more gas discharge nozzles being operative to cause a pressure drop in the gas flow therethrough such that the pressure of the gas discharged from the one or more gas discharge nozzles is of a second magnitude. The first magnitude is at least twice the second magnitude, thereby causing a shock wave in the gas and liquid flow downstream of the one or more gas discharge nozzles and one or more liquid discharge nozzles. The shock wave atomizes the liquid discharged from the one or more liquid discharge nozzles into a high velocity mist of droplets, thereby forming a mist of droplets suspended in the flow of the discharged high velocity gas.

In another embodiment of the method, the method further includes the step of grounding the pre-selected tissue so that the accelerated electrically charged droplet mist discharges when alighting on the pre-selected tissue.

In yet another aspect of the present invention there is provided a method for introducing a therapeutic substance into a patient. The method includes the steps of:

abrading a pre-selected area of dermal tissue, the abrasion being effected by liquid droplets of a high velocity liquid-gas mist;

applying electrical charge to the pre-selected area of dermal tissue thereby to electroporate the tissue, the charge being conveyed by the liquid droplets in the high velocity liquid-gas mist; and

bringing the therapeutic substance to the electroporated area of tissue for transdermal delivery therethrough.

In an embodiment of the method for introducing a therapeutic substance into a patient, the method further includes the steps of:
a) accelerating a flow of a gas at an elevated velocity;
b) introducing into the elevated velocity gas flow a flow of electrically charged liquid, thereby to fragment the liquid into a mist of electrically charged droplets, and to accelerate the mist to an accelerated velocity similar to the velocity of the gas flow; and
c) exposing the pre-selected area of dermal tissue to the accelerated electrically charged droplet mist.

In another embodiment of the method for introducing a therapeutic substance into a patient, the charge provided to the liquid is provided at a current of about 100 microamperes or less. In yet another embodiment of the method for introducing a therapeutic substance into a patient, the charge is delivered in pulses.

In a still another embodiment of the method for introducing a therapeutic substance into a patient, the step of bringing further includes the step of introducing the therapeutic substance into the liquid of a liquid source, the liquid source providing liquid for the liquid-gas mist so that the therapeutic substance is brought to the electroporated tissue by the liquid droplets of the mist.

### BRIEF DESCRIPTION OF THE DRAWINGS

The present invention will be more fully understood and its features and advantages will become apparent to those skilled in the art by reference to the ensuing description, taken in conjunction with the accompanying drawings, in which:

FIG. 1 is a block diagram of the system of the present invention;

FIG. 2 is a schematic presentation of an electroporation system constructed and operative in accordance with a first embodiment of the present invention;

FIG. 3 is a schematic side view of the system in FIG. 2;

FIGS. 4 and 5 are schematic side views of a delivery nozzle arrangement of the stream generating device (handpiece) of the system shown in FIG. 3;

FIG. 6A is a schematic presentation of an electroporation system constructed and operative in accordance with a second embodiment of the present invention;

FIG. 6B is a schematic presentation of an embodiment of the nozzle arrangement region of the stream generating device (handpiece) of the embodiment of the present invention shown in FIG. 6A;

FIGS. 7A and 7B are block diagrams in increasing detail of an exemplary electrical circuit for use in a system constructed and operative in accordance with an embodiment of the present invention;

FIG. 7C is a detailed presentation of the electrical circuit described in conjunction with FIGS. 7A and 7B; and

FIG. 8 is a graphical presentation of an oscillogram representing the electrical pulses generated by the discharging droplets of the present invention.

Similar elements in the Figures are numbered with similar reference numerals.

### DETAILED DESCRIPTION OF THE PREFERRED EMBODIMENTS

The system for electroporation of the present invention supplies supersonic flows of compressed gas and liquids to remove the outer layer(s) of tissue at the site of electroporation while also delivering high voltage electric charge to the liquid to effect electroporation. Such electroporation allows inter alia delivery of transdermal supplements, such as drugs. Abrading and moistening the tissue to be electroporated enhances the efficacy of the electroporation procedure because both processes lower tissue resistance.

The droplets emitted from the stream generating device, also often denoted herein as the handpiece, are charged with electricity and they behave like small capacitors. The droplets maintain their individuality and distribution in space between the nozzle of the handpiece and the tissue surface where electroporation takes place. The discharge of electricity from the droplets occurs when the droplets interact with the surface of the tissue. Discharge requires approximately the same duration of time as the time required for the droplets' contact with the tissue and subsequent deformation. As a result, the stream of charged droplets acts as a generator of stochastic wide-frequency current for providing high voltage electrodeless electroporation. The phrase “electrodeless electroporation” indicates that no electrode is attached to the area of tissue being treated as it is in other electroporation devices and systems. The tissue perceives the discharge of the droplets as electrical pulses.

As noted above, the supersonic flows remove dead tissue, contamination and bioburden, while intensively stimulating underlying tissue systems. The flow of accelerated micro-droplets causes exfoliation of the outer layers of tissue while concurrently applying static pressure and exposing the treatment site to the dynamic stochastic pressure of the accelerated droplets. Due to differences in pressure on the tissue surface and within the tissue itself, diffusion processes are intensified resulting in supplemental perfusion.

The droplets of liquid are accelerated, often to supersonic speeds, in the gap between the nozzle of the stream generating device, i.e. the handpiece, and the tissue surface. In this gap, the droplets are speeded up, change shape, disassemble, and become smaller. At the tissue surface, the droplets collapse, are channeled off and are whirled away.

The duration of interaction of droplets with the tissue surface is a function of numerous parameters, including, but not limited to, the droplets' dimension, speed, viscosity, surface tension, gas flow velocity, and tissue roughness and shape. Many droplets strike the tissue concurrently over a small area and their properties are distributed stochastically because of the parameters mentioned above. The tissue perceives its interaction with the discharging droplets as electrical pulses.

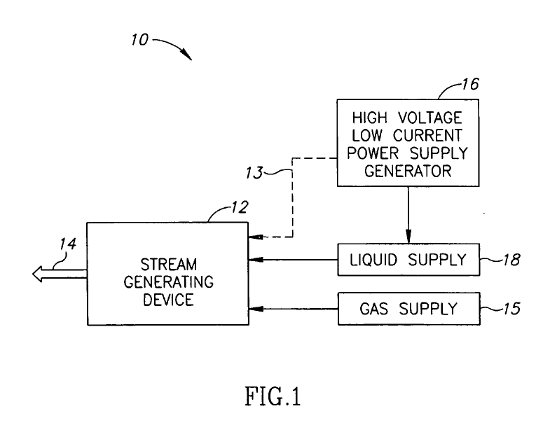

Referring now to FIG. 1, there is shown a block diagram of a system, generally referred to as 10, for applying a high velocity liquid-gas stream to tissue for treatment thereof. System 10 employs a stream generating device 12 similar to that disclosed in pending US application, “A High Velocity Liquid Gas Mist Tissue Abrasion Device”, Ser. No. 10/584760, filed on Jun. 27, 2006, to the present inventor and to that disclosed in pending US application, “A High Velocity Liquid-Gas Stream Device For Administering Therapeutic Substances”, Ser. No. 11/920412, filed on Nov. 14, 2007, also to the present inventor. Both applications are included herein by reference. These applications have also been published as International Patent Application Number PCT/IL2005/000017 and International Patent Application Number PCT/IL2006/000557, respectively.

System 10 is fed by a high pressure gas supply 15 and a liquid supply 18 and produces a high-velocity liquid-gas mist stream 14 containing electrostatic charge suitable for electroporation as described below. The present invention further includes a high voltage, low current generator 16 which is operative to introduce an electrostatic charge into liquid supply 18 so that the resulting high-velocity liquid-gas stream 14 includes electrostatically charged droplets which are applied to a tissue mass exposed to stream 14. Alternatively, generator 16 introduces electrostatic charge directly into stream generating device 12 as indicated by broken line 13.

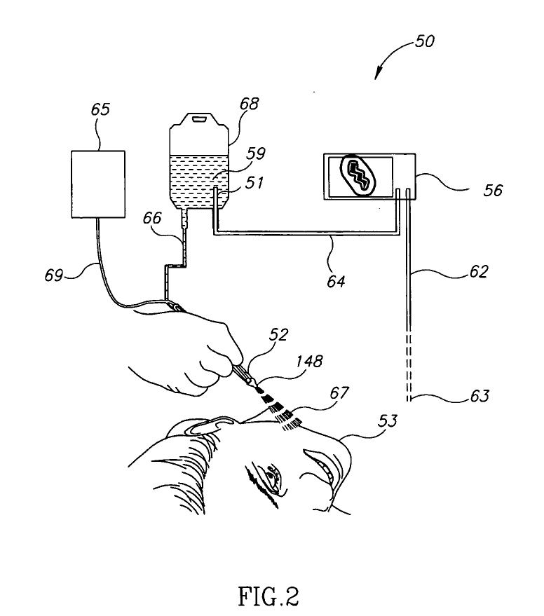

Reference is now made to FIG. 2 where a schematic representation of a first embodiment of the electroporation system of the present invention is shown.

System 50 includes a high voltage, low current electrical generator 56 in electrical connection via wire 64 to an electrode 51. Electrode 51 is positioned in a container 68 which contains an electrolytic solution 59 which can conduct electricity. Typically, but without intending to limit the invention, the solution may be a saline solution (NaCl). Electrostatic charge is transferred from generator 56 to solution 59 via wire 64.

Via probe 63, wire 62 electrically connects generator 56 with patient 53. The actual attachment to the patient of probe 63 is not shown and may be effected by using any of many known methods such as clips or attachment means similar to those used with electrocardiographs.

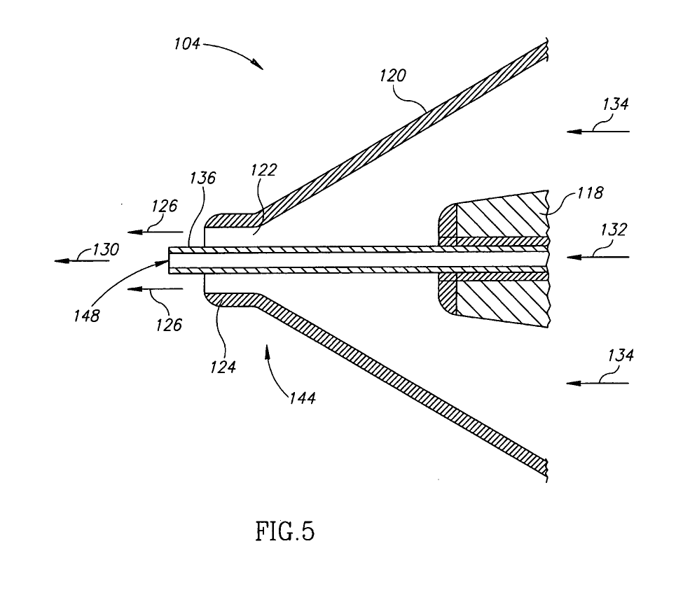

The electrolytic solution 59 is transferred from container 68 to handpiece 52 via conduit tube 66 under the negative pressure produced by the gas flow in the turbulence zone extending beyond the distal end of needles of handpiece 52. The needles are best seen in FIGS. 4 and 5 discussed below. Handpiece 52 is in pneumatic communication via tube 69 with a compressed gas source 65. The compressed gas that source 65 provides acts as a carrier and accelerator of charged liquid droplets emitted through the orifice 148 of handpiece 52.

The compressed gas typically, but without being limiting, may be supplied from the pressurized gas source at a pressure in the range of 40-150 p.s.i. The gas supplied from the pressurized gas source typically, but without being limiting, includes one or more gases selected from among air, oxygen, carbon dioxide and nitrogen.

The charged electrolytic solution 59 exits handpiece 52 as a stream/mist of charged droplets 67 from an orifice 148 on the end of handpiece 52 distal from conduit tube 66. Each droplet carries charge which when encountering the skin of patient 53 discharges causing high voltage electrodeless electroporation of the skin.

Upon emission of the solution as a liquid mist from orifice 148 of handpiece 52, further disassembly and fragmentation of the liquid occurs. The nascent droplets have the same charge as the charged electrolytic solution but as should be evident to those skilled in the art, the amount of electricity each droplet carries is inter alia a function of the electrical properties of the liquid, e.g. its dielectric strength, and the droplet's volume.

The charged droplets carry electrical charge to the tissue surface to be electroporated where the charge carried by the droplets is discharged. As noted above, the patient is in electrical communication via probe 63 and connecting wire 62, with high voltage power supply generator 56 forming a closed electrical circuit.

The tissue is moistened by the droplets improving its electrical conductivity. Exfoliation of the outer tissue layers by the flow of liquid-gas mist, often, but without intending to limit the invention, supersonic flow, also reduces tissue resistance and promotes transfer of the electrical charge. Because the aerodynamic liquid-gas flow is stopped at the tissue surface, static pressure on the tissue surface increases leading to increased liquid diffusion inside the tissue.

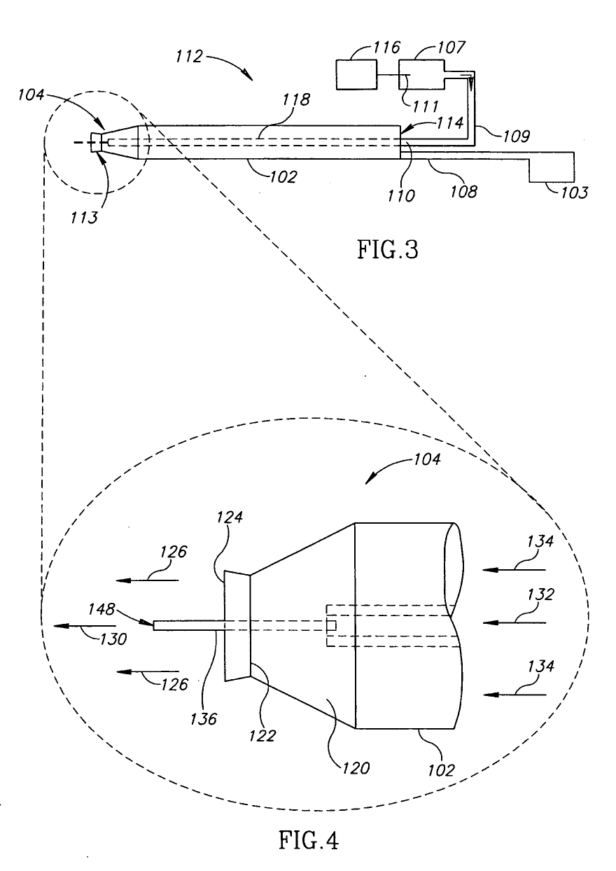

With reference to FIG. 3, there is seen, according to an embodiment of the present invention, a stream generating device referenced generally 112 for applying a high velocity liquid-gas mist to tissue thereby to cause abrasion, debridment, cleansing and electroporation thereof. As noted above, device 112 may also be referred to herein as handpiece 112. Stream generating device 112 includes a housing portion referenced 102 having a generally tubular configuration, and having proximal and distal ends, referenced generally 114 and 113, respectively. A gas inlet port referenced 108 and a liquid inlet port referenced 110 are provided at proximal end 114, and a mist jet delivery nozzle arrangement referenced generally 104, is provided at distal end 113. A compressed gas source 103 is in pneumatic communication with gas inlet port 108. Liquid supply 107 provides liquid to device 112 via conduit 109 which is in flow communication with liquid inlet port 110. An electrode 111 positioned in liquid supply 107 provides electric charge generated by high voltage low current generator 116 to the liquid in liquid supply 107.

Referring now to FIGS. 4 and 5 in conjunction with FIG. 3, there are seen schematic cross-sectional views of nozzle arrangement 104 of stream generating device 112. Nozzle arrangement 104 includes a gas discharge nozzle, referenced generally 144, and, disposed generally concentrically there-within, is a liquid discharge nozzle referenced 136. Liquid inlet port 110 is connected in fluid flow communication with liquid discharge nozzle 136 by means of a liquid communication tube referenced 118, disposed generally concentrically within tubular housing portion 102.

Pressurized gas supplied from pressurized gas source 103 enters stream generating device 112 through gas inlet port 108 and passes along and within tubular housing portion 102 as indicated by arrows 134, so as to discharge through gas discharge nozzle 144. Typically, but without intending to limit the invention, pressurized gas source 103 is held at a pressure in the range of about 40-150 psi. Gas discharge nozzle 144 is generally configured having, in flow succession, a converging portion referenced 120, a throat portion referenced 122 and a diverging discharge portion referenced 124. The pressurized gas discharging from nozzle 144, as indicated by arrows 126, undergoes a rapid and substantial reduction in pressure to atmospheric pressure and a substantial acceleration to a high velocity, within the range of subsonic to supersonic velocity and specifically to a supersonic velocity. Gas discharge nozzle 144 is configured such that the discharging gas has a cone angle of less than 10 degrees, which allows for a substantially parallel gas flow.

Liquid from liquid source 107 enters device 112 through liquid inlet port 110 via conduit 109 and passes, as indicated by arrow 132, through liquid communication tube 118. In turn, at distal end 113, liquid is discharged through an opening referenced 148 in the distal end of liquid discharge nozzle 136 into the discharging flow 126 of gas, the liquid flow being indicated by arrow 130.

It will be appreciated by persons skilled in the art that, as the pressurized discharging gas emerges 126 from gas discharge nozzle 144 into the atmosphere, it undergoes a rapid drop in pressure to atmospheric pressure. The sudden pressure drop results in a substantial acceleration of the velocity of the discharging gas flow that approximates or even exceeds the velocity of sound and the production of a shock wave. The effect of the shock wave is to atomize the liquid discharging from liquid discharge nozzle 136 into the flow of gas as a mist of liquid droplets 130, such that there is obtained a relatively narrow jet of liquid droplets in a high velocity gas flow 126.

Further, by way of example, the proportion of liquid flow to gas flow is extremely low due to the relatively high gas pressure of about 100 p.s.i. and low liquid pressure of about 2 p.s.i., as well as the relatively large internal diameter of gas discharge nozzle 144 (about 0.8 mm) compared to a small internal diameter (about 0.09 mm) of liquid discharge nozzle 136. Consequently, little liquid tends to accumulate at the site to be abraded or scoured. Furthermore, the relatively high gas flow has the effect of dispersing any accumulated liquid. When using a jet utilizing only liquid for cleansing, the liquid tends to accumulate on the tissue surface resulting in formation of a virtually stagnant liquid boundary layer close to and in contact with the surface, thereby reducing the effectiveness of cleansing.

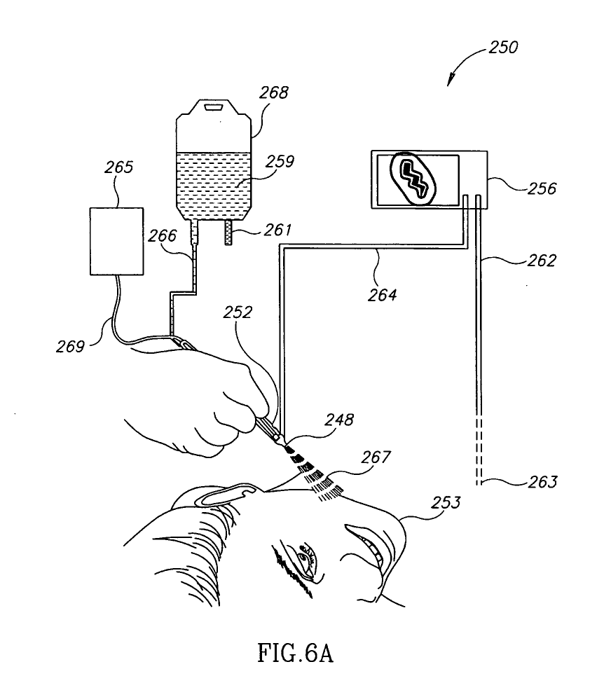

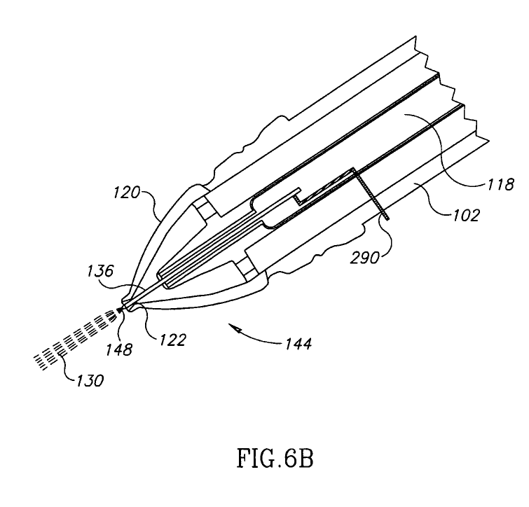

Reference is now made to FIG. 6A where a schematic representation of a second embodiment of the electroporation system of the present invention is shown. The present embodiment is very similar to the one shown in FIG. 2 with the exception that the charge provided by the generator is transferred to the liquid within, or at the distal end of, handpiece 252. This embodiment is shown in the block diagram of the system in FIG. 1 as broken line 13.

System 250 includes a high voltage, low current electrical generator 256 in electrical connection via wire 264 to an electrode (not shown) positioned in handpiece 252. Electrostatic charge is transferred from generator 256 to the liquid as it passes through handpiece 252. In some embodiments, the electrode may be positioned adjacent to the orifice 248 of handpiece 252, charging the liquid as it exits orifice 248 of handpiece 252.

Via probe 263, wire 262 electrically connects generator 256 with patient 253. The actual attachment of probe 263 to the patient is not shown and may be effected by using any of many known methods, such as clips or attachment means similar to those used with electrocardiographs.

Electrolytic solution 259 is transferred from container 268 to handpiece 252 via conduit tube 266 under the negative pressure produced by the gas flow in the turbulence zone extending beyond the distal end of the needles of handpiece 252. The needles are best seen in FIG. 6B. Electrolytic solution container 268 may include a port 261 through which one or more therapeutic substances, such as one or more medicinal drugs, may be introduced into a patient via the electroporation process described herein. Handpiece 252 is in pneumatic communication via tube 269 with a compressed gas source 265. The compressed gas that source 265 provides acts as a carrier and accelerator of charged liquid droplets emitted through the orifice of the handpiece.

The charged electrolytic solution 259 exits handpiece 252 as a stream/mist of charged droplets 267 from orifice 248 on the end of handpiece 252 distal from conduit tube 266. Each droplet carries charge which when encountering the skin of patient 253 discharges causing high voltage electrodeless electroporation of the skin.

As noted above in regard to the embodiment shown in FIG. 2, upon emission of the solution as a liquid mist 267 from orifice 248 of handpiece 252, further disassembly and fragmentation of the liquid occur. Each of the nascent droplets carries an amount of electrical charge which is a function of the electrical properties of the liquid, e.g. dielectric strength, and the droplet's volume.

Again as noted above in regard to the embodiment shown in FIG. 2, the charged droplets carry electrical charge to the tissue surface to be electroporated where the charge carried by the droplets is discharged. As noted above, the patient is in electrical communication via probe 263 and connecting wire 262, to high voltage power supply 256 which closes the electrical circuit.

Dynamic pressure of the flow is restored due to interaction between the liquid-mist flow and the tissue surface. The tissue is moistened improving its electrical conductivity. Exfoliation of the outer tissue layers by the flow, often supersonic flow, of the liquid gas mist also reduces tissue resistance and promotes transfer of the electrical charge.

Referring now to FIG. 6B, there is shown a schematic representation of the distal end of handpiece 252 presented in FIG. 6A. In this embodiment the electrode for charging the liquid is positioned within handpiece 252.

FIG. 6B shows the distal end of handpiece 252. An electrode 290 is positioned within tubular housing 102 and inside liquid discharge nozzle 136. The remaining elements in the Figure (and their operation) have been discussed previously. Accordingly, liquid communication tube 118, converging portion 120, throat portion 122, gas discharge nozzle 144, and orifice 148, which are constructed and operative to act as discussed above to produce a liquid gas mist 130 will not be discussed again.

The present invention also contemplates that the devices and systems taught herein may include a plurality of gas discharge nozzles and/or a plurality of liquid discharge nozzles rather than a single gas discharge nozzle and a single liquid discharge nozzle.

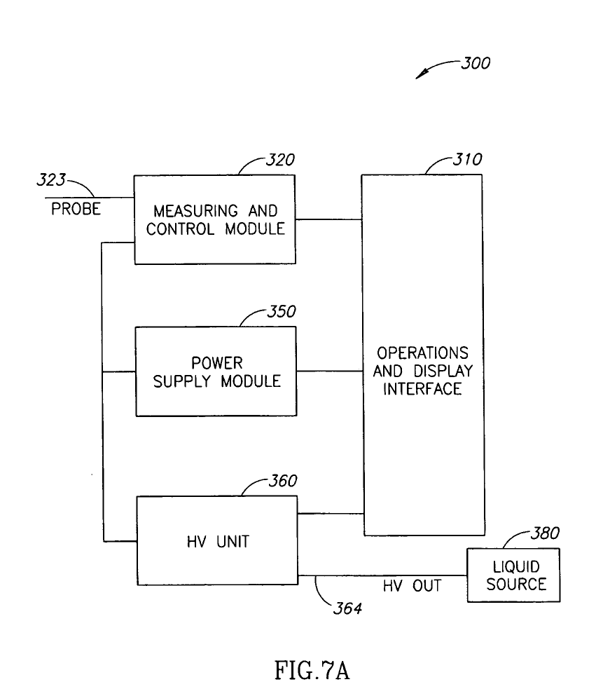

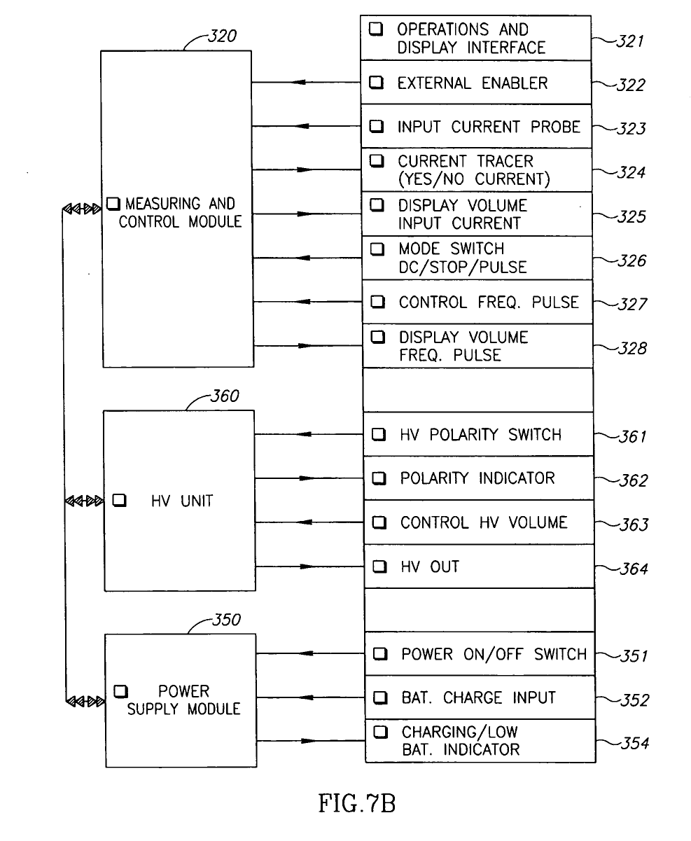

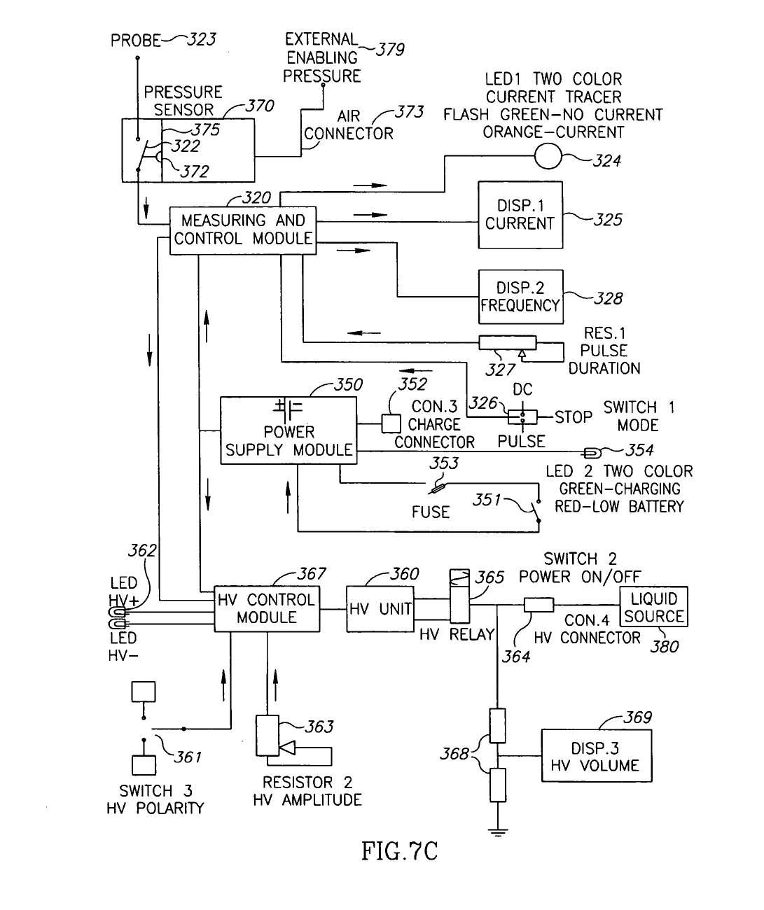

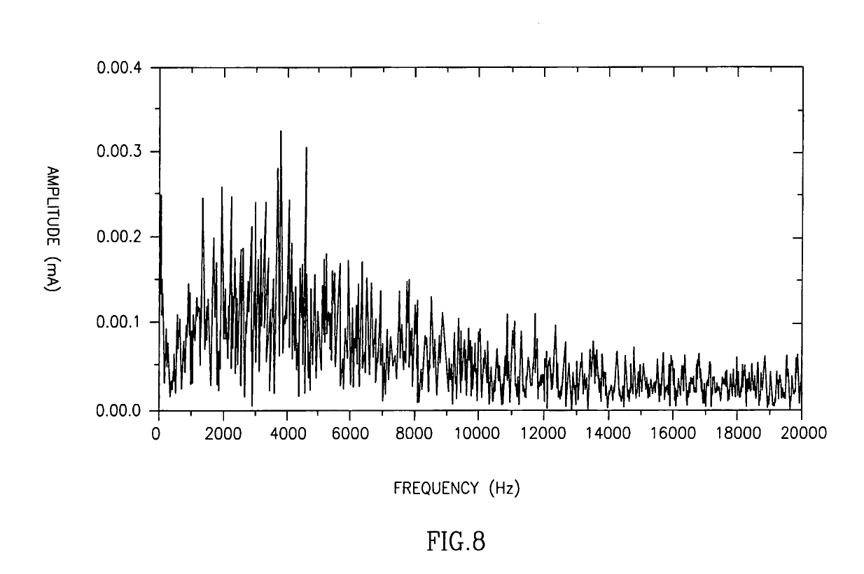

FIGS. 7A and 7B, to which reference is now made, are block diagrams of an exemplary electrical circuit for an embodiment of the system of the present invention. FIG. 7B is a more detailed block diagram than FIG. 7A.

FIG. 7A shows a block diagram of an electrical circuit 300 and supporting electronics containing a measuring and control module 320 in electronic communication with a power supply module 350 and a high voltage (HV) unit 360. Modules 320 and 350 and unit 360 are each in communication with an operations and display interface 310. Measuring and control module 320, in addition to activating and controlling power supply module 350 and HV unit 360, is in communication with a probe 323. The probe is in contact with the patient. The HV module is also connected via an HV out connector 364 to an electrode in an electrolytic solution which supplies charged liquid to the handpiece and then to the tissue being treated. Alternatively, connector 364 may be in electrical communication with an electrode positioned in the electrolytic solution passing through the handpiece of an electroporation system as described in conjunction with FIGS. 6A and 6B.

FIG. 7B is a more detailed block diagram than FIG. 7A. FIG. 7A shows the modules presented in FIG. 7A and their display interface 310 in electrical communication with other elements of the exemplary electrical circuitry. As presented in FIG. 7B, the power supply module 350 inter alia is in electrical communication with an on/off power switch 351, a battery charge input 352, and a battery chargeing/ battery low indicator 354. The HV unit 360 inter alia is in electrical communication with an HV polarity switch 361, a polarity indicator 362, an HV volume control 363, and an HV out line 364. The measuring and control module 320 inter alia is in communication with an external enabling pressurized gas source 322, element 379 in FIG. 7C, an input current probe 323, a current tracer 324, an input current volume display 325, a mode switch 326, a pulse frequency control 327 and a pulse frequency volume display 328.

FIG. 7C shows in even greater detail the exemplary electrical circuit and supporting electronics schematically presented in FIGS. 7A and 7B and the relationship of the elements in the circuit to each other.

Power supply module 350 is in electrical communication with and activated by an on/off switch 351. When required, switch 351 may be disconnected from power supply module 350 by a fuse 353. A battery in power supply module 350 is charged via battery charge connecter 352. An LED 354 is in electrical communication with power supply module 350 and indicates if the battery in module 350 is charging or running low. Power supply module 350 provides current to HV control module 367 and to measuring and control module 320.

HV control module 367 is in electrical communication with an HV unit 360 where a pulsed high voltage current is generated. The HV control module 367 is also in electrical communication with an HV polarity switch 361 and LEDs 362, the latter indicating the polarity status of HV unit 360 and the former used for selecting the HV polarity of the HV unit. HV control module 367 is also in electrical communication with a variable resistor 363 which controls the HV amplitude. The HV low current provided by HV unit 360 is transferred to an HV relay 365. HV relay 365 is in electrical communication with an HV display 369. It is also electrically grounded after passing through resistors 368. Similarly, HV relay 365 is in communication with an HV out connector 364 which in turn is in electrical communication via an electrode with liquid passing through the handpiece of the system or directly with liquid in a liquid supply 380 which provides liquid to a handpiece.

Measuring and control module 320 is in electrical communication with a variable resistor 327 which determines the duration of an electrical pulse generated by HV unit 360. The determined pulse duration is provided by module 320 to HV control module 367 and from there to HV unit 360. Module 320 is also controlled by a three-way switch 326 which determines the mode of the circuit: pulsed, DC or stop, i.e. off. Measuring and control module 320 is in communication with an LED 324 which functions as a current tracer indicating whether current is or is not passing through the module.

As indicated above, the exemplary electrical circuit exhibits pulses, that is it sends charge at pre-defined intervals for pre-defined durations to the liquid sent through the handpiece of the system. This periodic charging leads to pulses of charged droplets alighting on the tissue. This results in the droplet discharge being of a pulsed nature similar in duration to, and temporally tracking, the pulses of electricity used to charge the liquid.

Measuring and control module 320 also electrically communicates with a current display 325 and a frequency display 328 on a display interface (not shown here, but shown as element 321 in the block diagram of FIG. 7B). It should be noted that HV volume display 369, HV polarity LEDs 362, charging LED 354 and current tracer LED 324 also may be positioned on the display interface.

Measuring and control model 320 is also in communication with a pressure sensor 370 via a pressure inlet 372 and a pressure switch 322. Pressure sensor 370 using pressure sensitive membrane 375 determines the air pressure being provided to the system by an external enabling pressurized gas source 379 via air connector 373. The status of the pressurized gas as reflected by pressure sensor 370 is communicated to HV unit 360 via measuring and control module 320. Because of safety considerations, HV unit 360 is only operative when pressurized gas is being supplied as indicated by pressure sensor 370. Pressure sensor 370 is also in communication with probe 323 which is in contact with the patient.

HV control module 367, HV unit 360, HV relay 365 and power supply module 350 as well as all the other elements of the circuit in FIG. 7C are readily available commercially from many different suppliers.

It should be evident to those skilled in the art that the electrical circuitry described in conjunction with FIGS. 7A, 7B and 7C is exemplary only. It is not to be deemed as limiting the design of the electrical circuitry for the invention. As should be evident to one skilled in the art, other suitable electrical circuits may be designed for use with the systems taught and contemplated by the present invention.

Two kinds of pulse generators have been used in prior art to effect electroporation and to increase the transport of molecules through electroporized tissue. There are generators which can deliver exponentially decaying pulses or square wave pulses. Both have been used for increasing the rate of drug delivery.

The main advantage of exponentially decaying pulses results from its long high voltage tail. This maintains the high permeability state of the tissue induced by electroporation over long periods of time, promoting increased electrophoretic movement. However, because the duration of exponential decay pulses depends on the resistance of the tissue being electroporized and the electroporation system itself, e.g. electrodes, conducting medium, etc., the reproducibility of the pulse conditions is often problematic. This makes such pulses less desirable for clinical use.

In contrast, the voltage and duration of square wave pulses remain constant regardless of the nature and condition of the tissue being treated or the nature of the conducting liquid medium or the electrodes being used. Hence, square wave pulses provide better control and reproducibility when employed for enhancing drug transport. Until recently, square wave pulses were exclusively used for electrochemotherapy and DNA electrotransfer, whereas exponentially decaying pulses were restricted to transdermal drug delivery to take advantage of the long voltage tail.

Pulses in prior art systems arise from a power supply that forms and sends HV pulses via wires and electrodes to the tissue that is to undergo electroporation. The initial form of the pulses is, or may be, modified on its way to the tissue. In the present invention, pulse formation occurs only at the moment of contact between a droplet and the tissue. The electrified droplets can be thought of as autonomous microscopic power supplies.

A typical graph of discharged current is shown in FIG. 8 to which reference is now made. The graph shows current amplitude as a function of discharge frequency. FIG. 8 reflects the droplet discharge process, a process that effectively generates small electrical pulses. The discharge reflects a typical pulse generated by an electrical circuit essentially similar to the one shown in FIG. 7C.

The graph shown in FIG. 8 was obtained using a system such as that presented in FIG. 2. The orifice of the handpiece was 1 mm. in diameter. The air supply and water supply pressures were 90 psi and 3 psi, respectively. The system was operated at a voltage of 1 kV. The current supplied was an integrated current of discharging droplets (shown on the vertical axis of FIG. 8) while the pulse duration used in producing FIG. 8 was a value inversely related to the frequencies (horizontal axis of FIG. 8). The droplet dimensions exhibited a Gaussian distribution from a maximum of about 200 microns to a minimum of about 1 micron. The integrated current in FIG. 8 is a Gaussian curve similar to the one representing the distribution of the droplet dimensions. The sensing electrode, the electrode which simulates the tissue in in vivo applications of the present invention, had a diameter of 3 mm; the orifice of the gas nozzle had a diameter of about 0.5 mm. The sensing electrode was connected to a power supply and was placed 10 mm in front of the nozzle of the handpiece.

In this measurement, as in general, at any moment many different droplets discharge on the electrode surface creating a current with a marked similarity to a direct current (DC). As noted above, the surface of the sensing electrode here simulated a tissue surface in an in vivo setting. The current consists of many individual droplets discharging on the electrode surface over time, creating a complex interaction of electrical discharges, each droplet's discharge adding to those of previous droplet discharges. As noted above, the discharging droplets reflect electrical pulses.

Because of safety considerations, the systems of the present invention are typically constructed to provide charge at currents of no more than about 100 microamperes. As noted above, the range of pulses generated is dependent inter alia on the number and dimensions of the mist droplets generated. The voltages used may be about 6 kV However, it should be noted that while there is no theoretical constraint on the maximum voltage that can be used, there are practical considerations such as cost and safety which limit the actual applied voltage.

A method for electroporation of a pre-selected area of tissue is provided in the present invention. The method includes the following steps:
a) accelerating a flow of a gas to an elevated velocity;
b) introducing into the elevated velocity gas flow a flow of electrically charged liquid, fragmenting the liquid into a mist of electrically charged droplets, and accelerating the mist to an accelerated velocity similar to the velocity of the gas flow; and
c) exposing the pre-selected area of tissue to the accelerated electrically charged droplet mist.

The method may further include the step of providing charge to the liquid. The charge may be provided at any stage prior to its introduction into the gas flow.

The method may further include the step of discharging the charge of the accelerated electrically charged droplet mist when the droplets alight on the pre-selected tissue.

The method may further include the step of grounding the tissue so that the charge of the accelerated electrically charged droplet mist is discharged when alighting on the pre-selected tissue.

In the method, the charge supplied is at a current of about 100 microamperes or less.

In another aspect of the present invention there is provided a method for introducing a therapeutic substance into a patient. The method includes the steps of:
abrading a pre-selected area of dermal tissue to decrease the contact resistance of the tissue, the abrasion being effected by a high velocity liquid-gas mist;
applying electric current to the pre-selected area of dermal tissue thereby to electroporate the tissue, the current being conveyed by the liquid droplets in the high velocity liquid-gas mist; and
bringing the therapeutic substance to the electroporated area of tissue for transdermal delivery therethrough.

In an embodiment of the method for introducing a therapeutic substance into a patient, the method further includes the steps of:
a) accelerating a flow of a gas at an elevated velocity;
b) introducing into the elevated velocity gas flow a flow of electrically charged liquid, fragmenting the liquid into a mist of electrically charged droplets, and accelerating the mist to an accelerated velocity similar to the velocity of the gas flow; and
c) exposing the pre-selected area of dermal tissue to the accelerated electrically charged droplet mist.

In the method for introducing a therapeutic substance into a patient, the charge applied is at a current of about 100 microamperes or less.

In the method for introducing a therapeutic substance into a patient, the charge is delivered in pulses.

In a still another embodiment of the method for introducing a therapeutic substance into a patient, the method may further include the step of introducing the therapeutic substance into the liquid of a liquid source. The liquid source supplies liquid for the liquid-gas mist so that the therapeutic substance is brought to the electroporated tissue by the liquid droplets of the mist.

Advantages of the electroporation devices and systems of the present invention are:
There is no need for an electrode(s) to be physically in contact with the tissue being electroporated as there is with other electroporation systems.
Droplet discharging and resulting electroporation covers larger tissue areas than prior art electroporation methods.
There is substantially simultaneous (a) tissue exfoliation with a resultant reduction in tissue electrical resistance, (b) tissue cell electroporation, and (c) treatment supplement delivery and infusion.
A wide range of values for the various electrical pulse parameters may be used. These parameters include, but are not limited to, pulse amplitudes, frequencies and voltages.

It should be readily apparent to one skilled in the art that the device and method of the present invention can be used in procedures on animals, particularly mammals, as well as on humans.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present invention.

It will be appreciated by persons skilled in the art that the present invention is not limited by the drawings and description hereinabove presented. Rather, the invention is defined solely by the claims that follow.

What is claimed is:

1. A device for electroporation of pre-selected tissue, which includes:
a) a mist jet delivery nozzle arrangement having a distal end and a proximal end and connected to a pressurized gas source and also connected to a liquid source; and
b) a charge generator in electrical communication with the liquid provided by said liquid source wherein said generator supplies charge to the liquid, and
wherein said delivery nozzle arrangement is arranged and operative to accelerate and emit the pressurized gas and arranged and operative to similarly accelerate and emit the liquid as a mist of accelerated charged droplets, the charged droplets alighting and then electrically discharging on the pre-selected tissue effecting electroporation.

2. A device in accordance with claim 1 wherein said charge generator provides charge to the liquid at a current of about 100 microamperes or less.

3. A device according to claim 1 wherein said mist jet delivery nozzle arrangement further includes:
i) at least one gas discharge nozzle arranged to receive a flow of pressurized gas from said gas source via a gas inlet port and said at least one gas discharge nozzle configured to accelerate the flow of gas so as to emit it at an elevated velocity; and
ii) at least one liquid discharge nozzle arranged to receive a flow of liquid from said liquid source via a liquid inlet port and operative to emit the flow of liquid into the elevated velocity flow of gas, thereby to similarly accelerate the velocity of the discharged liquid as a mist of accelerated droplets.

4. A device in accordance with claim 3 wherein the flow of gas entering said at least one gas discharge nozzle is at a pressure of a first magnitude, and said at least one gas discharge nozzle is operative to cause a pressure drop in the gas flow therethrough such that the pressure of the gas emitted from said at least one gas discharge nozzle is of a second magnitude, wherein the first magnitude is at least twice the second magnitude, so as to cause a shock wave in the gas and liquid flow downstream of said at least one gas discharge nozzle and said at least one liquid discharge nozzle so as to cause atomizing of the liquid emitted from said at least one liquid discharge nozzle into a high velocity mist of droplets, thereby to form a mist of droplets suspended in the flow of emitted high velocity gas.

5. A device according to claim 1 wherein said device further includes an electrode positioned in said liquid source for charging the liquid therein with charge supplied by said charge generator.

6. A device according to claim 1 wherein said device further includes an electrode positioned in said mist jet delivery nozzle for charging the liquid being delivered thereto and emitted therefrom, said electrode being in electrical communication with said charge generator.

7. A system for electroporation of pre-selected tissue of a patient, which includes:
a) a pressurized gas source;
b) a liquid source; and
c) a device which includes:
i) a gas inlet port connected to said pressurized gas source;
ii) a liquid inlet port connected to said liquid source;
iii) a mist jet delivery nozzle arrangement having a distal end and a proximal end and including:
1) at least one gas discharge nozzle arranged to receive a flow of pressurized gas from said gas inlet port and configured to accelerate the flow of gas so as to emit it at an elevated velocity; and
2) at least one liquid discharge nozzle arranged to receive a flow of liquid from said liquid inlet port and operative to emit the flow of the liquid into the elevated velocity flow of gas at said distal end of said nozzle arrangement, thereby to similarly accelerate the emitted liquid as a mist of accelerated droplets;
iv) a charge generator in electrical communication with the liquid so that the accelerated droplets carry charge supplied by said generator; and
v) an electrical connector in electrical communication with said charge generator and attachable to the body of the patient,
said system thereby allowing for the discharge of the electrically charged droplets when the droplets alight on the pre-selected tissue effecting electroporation therein.

8. A system according to claim 7 wherein said device further includes an electrode positioned in said liquid source for electrically charging the liquid therein with charge supplied by said charge generator.

9. A system according to claim 7 wherein said device further includes an electrode positioned in said mist jet delivery nozzle arrangement for charging the liquid being delivered thereto and emitted therefrom, said electrode being in electrical communication with said charge generator.

10. The system according to claim 7 wherein the flow of gas entering said at least one gas discharge nozzle is at a pressure of a first magnitude, and said at least one gas discharge nozzle is operative to cause a pressure drop in the gas flow therethrough such that the pressure of the gas emitted from said at least one gas discharge nozzle is of a second magnitude, wherein the first magnitude is at least twice the second magnitude, so as to cause a shock wave in the gas and liquid flow downstream of said at least one gas discharge nozzle and said at least one liquid discharge nozzle so as to cause atomizing of the liquid emitted from said at least one liquid discharge nozzle into a high velocity mist of droplets, thereby to form a mist of droplets suspended in the flow of emitted high velocity gas.

11. A method for electroporation of a pre-selected area of tissue, said method including the steps of:
a) accelerating a flow of a gas to an elevated velocity;
b) introducing into the elevated velocity gas flow a flow of electrically charged liquid, thereby to fragment the liquid into a mist of electrically charged droplets, and to accelerate the mist to an accelerated velocity similar to the velocity of the gas flow; and
c) exposing the pre-selected area of tissue to the accelerated electrically charged droplet mist.

12. The method according to claim 11 wherein said method further includes the step of providing charge to the liquid.

13. The method according to claim 12 wherein the charge provided is at a current of about 100 microamperes or less.

14. The method according to claim 11 wherein said step of accelerating a flow of a gas includes accelerating the gas through at least one gas discharge nozzle so as to provide a gas flow at an elevated velocity.

15. The method according to claim 14 wherein said step of introducing into the elevated velocity gas flow a flow of an electrically charged liquid, includes the elevated gas flow being at a pressure of a first magnitude, and the at least one gas discharge nozzle being operative to cause a pressure drop in the gas flow therethrough such that the pressure of the gas discharged from the at least one gas discharge nozzle is of a second magnitude, wherein the first magnitude is at least twice the second magnitude, thereby causing a shock wave in the gas and liquid flow downstream of the at least one gas discharge nozzle and an at least one liquid discharge nozzle so as to cause atomizing of the liquid discharged from the at least one liquid discharge nozzle into a high velocity mist of droplets, thereby forming a mist of droplets suspended in the flow of the discharged high velocity gas.

16. The method according to claim 11 further including the step of grounding the pre-selected tissue so that the accelerated electrically charged droplet mist is discharged when alighting on the pre-selected tissue.

17. A method for introducing a therapeutic substance into a patient, said method including the steps of:
abrading a pre-selected area of dermal tissue, the abrasion being effected by liquid droplets of a high velocity liquid-gas mist;
applying electrical charge to the pre-selected area of dermal tissue thereby to electroporate the tissue, the charge being conveyed by the liquid droplets in the high velocity liquid-gas mist; and
bringing the therapeutic substance to the electroporated area of tissue for transdermal delivery therethrough.

18. A method according to claim 17, further including the steps of:
a) accelerating a flow of a gas at an elevated velocity;
b) introducing into the elevated velocity gas flow a flow of electrically charged liquid, thereby to fragment the liquid into a mist of electrically charged droplets, and to accelerate the mist to an accelerated velocity similar to the velocity of the gas flow; and
c) exposing the pre-selected area of dermal tissue to the accelerated electrically charged droplet mist.

19. A method according to claim 17, wherein the charge applied is at a current of about 100 microamperes or less.

20. A method according to claim 19, wherein the charge is delivered in pulses.

21. A method according to claim 17, wherein said step of bringing further includes the step of introducing the therapeutic substance into a liquid of a liquid source, the liquid source supplying the liquid for the liquid-gas mist so that the therapeutic substance is brought to the tissue undergoing electroporation by the liquid droplets of the mist.
